(19) United States
(12) Patent Application Publication
(10) Pub. No.: US 2023/0321415 A1
(43) Pub. Date: Oct. 12, 2023

(54) LUMEN INSERTABLE CAPSULE

(71) Applicant: Novo Nordisk A/S, Bagsvaerd (DK)

(72) Inventors: Brian Mouridsen, Fredensborg (DK); Drago Sticker, Malmoe (SE); Aghiad Ghazal, Koebenhavn SV (DK); Andre Larsen, Dragoer (DK); Jacob Pyung Hwa Jepsen, Frederiksberg (DK)

(21) Appl. No.: 18/025,347

(22) PCT Filed: Sep. 29, 2021
(86) PCT No.: PCT/EP2021/076891

§ 371 Date: Mar. 8, 2023

(30) Foreign Application Priority Data
Sep. 30, 2020 (EP) 20199486.0

(51) Int. Cl. A61M 31/00

(52) U.S. Cl. CPC A61M 31/002

(57) ABSTRACT

A capsule device (100) suitable for insertion into a lumen, such as a gastrointestinal lumen, of a subject. The capsule device (100) comprises: a capsule housing (110, 120), a drug chamber (B) configured to accommodate a drug substance, the drug chamber leading to a drug outlet (190),—an actuation chamber (A),—a movable separator (160) arranged between the actuation chamber and the drug chamber, wherein movement of the movable separator expels drug substance from the drug chamber (B) through the drug outlet (190), and—a gas generator (140, 150) configured actuatable to generate pressurized gas in the actuation chamber (A) for exerting mechanical load on the moveable separator (160) to expel the drug substance. A burst gate (170) is arranged between the gas generator (140, 150) and the movable separator (160), the burst gate (170) configured to release mechanical load onto the movable separator (160) upon increase in gas pressure in the actuation chamber (A) above a threshold pressure level to thereby initiate expelling of the drug substance.

The present invention relates to lumen insertable devices, such as ingestible capsules for delivery of a drug substance to a subject user.

### BACKGROUND OF THE INVENTION

In the disclosure of the present invention reference is mostly made to the treatment of diabetes by delivery of insulin, however, this is only an exemplary use of the present invention.

May people suffer from diseases, such as diabetes, which requires them to receive injections of drugs on a regular and often daily basis. To treat their disease these people are required to perform different tasks which may be considered complicated and may be experienced as uncomfortable. Furthermore, it requires them to bring injection devices, needles and drugs with them when they leave home. It would therefore be considered a significant improvement of the treatment of such diseases if treatment could be based on oral intake of tablets or capsules.

However, such solutions are very difficult to realise, since protein-based drugs will be degraded and digested rather than absorbed when ingested.

To provide a working solution for delivering insulin into the bloodstream through oral intake, the drug has to be delivered firstly into a lumen of the gastrointestinal tract and further into the wall of the gastrointestinal tract (lumen wall). This presents several challenges among which are: (1) The drug has to be protected from degradation or digestion by the acid in the stomach. (2) The drug has to be released while being in the stomach, or in the lower gastrointestinal tract, i.e. after the stomach, which limits the window of opportunity for drug release. (3) The drug has to be delivered at the lumen wall to limit the time exposed to the degrading environment of the fluids in the stomach and in the lower gastrointestinal tract. If not released at the wall, the drug may be degraded during its travel from point of release to the wall or may pass through the lower gastrointestinal tract without being absorbed, unless being protected against the decomposing fluids.

Capsule devices have been proposed for delivery of a drug substance into a lumen or lumen wall. After insertion of the capsule device, such as by swallowing the capsule device into the GI system of the subject, drug delivery may be performed using am actuator comprising a gas generator which forces the drug substance through an outlet. For certain types of drug delivery rapid delivery is believed to be beneficial but gas generation may not offer sufficient drive pressure in a timely manner.

WO 92/21,307 A1 discloses a telemetry capsule for release of medicaments in the alimentary canal of animals, particularly humans, wherein a gas generator comprising liquid and solid reactants is initially kept isolated by a pierceable diaphragm. Upon receipt of a remote trigger signal the diaphragm becomes ruptured to allow the reactants to mix and hence gas generation to be initiated thereby forcing medication from a medicament storage compartment towards an outlet.

WO 2018/049,133 A1 discloses various ingestible devices wherein some of these include a jet delivery mechanism for delivery through an outlet provided as a jet nozzle, and wherein a gas generating cell propels a piston to move towards the nozzle such that a dispensable substance can be pushed under gas pressure to break a burst disc arranged upstream from the jet nozzle. Further related disclosure of ingestible devices are provided in WO 2020/106,750 A1 and WO 2018/213,600 A1.

Having regard to the above, it is an object of the present invention to provide a lumen insertable capsule device which is improved with respect to reliability in operation and which in a safe manner enables pressurized gas to operate a release mechanism for initiating expelling of a drug substance.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in an aspect of the invention, a capsule device suitable for insertion into a lumen, such as a gastrointestinal lumen, of a human or animal subject is provided. The capsule device comprises:
a capsule housing,
a drug chamber configured to accommodate a drug substance, the drug chamber leading to a drug outlet, an actuation chamber,
a movable separator arranged between the actuation chamber and the drug chamber, wherein movement of the movable separator expels drug substance from the drug chamber through the drug outlet, and
a gas generator configured actuatable to generate pressurized gas in the actuation chamber for exerting mechanical load on the moveable separator to expel the drug substance,
   wherein a burst gate is arranged between the gas generator and the movable separator, the burst gate being configured to release mechanical load onto the movable separator upon increase in gas pressure in the actuation chamber above a threshold pressure level to thereby initiate expelling of the drug substance.

In solutions suggested in the prior art comprising a burst gate arranged downstream from the drive mechanism, the burst gate may need to be sufficiently spaced relative to the drug outlet in order to minimize potential blocking structures of the burst gate from interfering with the expelling through the drug outlet

In accordance with the invention, the inclusion of a burst gate arranged between the gas generator and the movable separator enables the release of pressurized gas in a controlled and safe manner. Compared with systems wherein a burst gate is arranged at the output side of the expelling system, e.g. in the vicinity of the drug outlet, the risk associated with potential loose fragments associated with operation of the burst gate for initiating expelling is reduced. Besides, arranging the burst gate disposed between the gas generator and the movable separator ensures that the system can be built in a particular space-saving manner. Furthermore, the burst gate will become isolated from the drug holding components avoiding or reducing potential interaction issues during long term storage between the drug substance and components of the burst gate.

In some embodiments, the actuation chamber comprises first and second actuation compartments being separated by the burst gate, wherein the gas generator supplies gas to the first actuation compartment, and wherein the second actuation compartment is in gas fluid communication with the movable separator.

In some forms the burst gate comprises or is provided as a rupturable membrane, such as a burst disc.

In other forms the burst gate may be formed to comprise a burst valve arrangement.

In some variants the burst valve arrangement comprises a bi-stable spring element being movable from a first stable position to a second stable position upon an increase in gas pressure in the first actuation compartment, wherein the bi-stable spring element releases mechanical load onto the movable separator when moving from the first stable position towards the second stable position.

In still further variants, the bi-stable spring element comprises a gas port which, when the bistable element assumes the first stable position, is maintained closed by a brittle and/or breakable material portion, and wherein the brittle and/or breakable material portion breaks when the bi-stable element is moved towards the second stable position to enable pressurized gas to flow through the gas port.

In some embodiments a trigger arrangement is comprised with the capsule device, the trigger arrangement being configured to actuate the gas generator.

In some variants the trigger arrangement comprises an environmentally-sensitive mechanism.

In some forms, the capsule device is configured for swallowing by a patient and travelling into a lumen of a GI tract of a patient, such as the stomach, the small intestine or the large intestine, respectively.

The environmentally-sensitive mechanism may in certain embodiments be a GI tract environmentally-sensitive mechanism. The GI tract environmentally-sensitive mechanism may comprise a trigger member, wherein the trigger member is characterised by at least one of the group comprising:
a) the trigger member comprises a material that degrades, erodes and/or dissolves due to a change in pH in the GI tract;
b) the trigger member comprises a material that degrades, erodes and/or dissolves due to a pH in the GI tract;
c) the trigger member comprises a material that degrades, erodes and/or dissolves due to a presence of an enzyme in the GI tract; and
d) the trigger member comprises a material that degrades, erodes and/or dissolves due to a change in concentration of an enzyme in the GI tract.

In further embodiments the gas generator associated with the actuation chamber comprises effervescent material and wherein the capsule housing comprises a fluid inlet portion leading to the effervescent material.

In some forms of the capsule device, the fluid inlet portion initially comprises an enteric coating adapted to dissolve when subjected to a biological fluid within the lumen, wherein biological fluid within the lumen is allowed to flow through the fluid inlet portion upon dissolving of the enteric coating to cause contact between the biological fluid and the effervescent material.

In further embodiments the fluid inlet portion comprises a semi-permeable membrane allowing biological fluid within the lumen to migrate through the semi-permeable membrane and enter into contact with the effervescent material.

In alternative embodiments, the capsule device comprises a liquid compartment filled with a liquid, wherein the gas generator comprises an effervescent material configured to generate gas when subjected to contact with liquid from the liquid compartment, and wherein operation of the trigger arrangement enables contact between the effervescent material and the liquid.

In still other embodiments, the gas generator comprises at least a first reactant and a second reactant configured to generate gas upon contact between the first reactant and the second reactant.

The movable separator may in some embodiment be provided as or comprise a piston which is arranged slidable in the drug chamber.

In some embodiments, the lumen, such as the small intestine, defines a lumen wall, wherein the drug outlet comprises a jet nozzle arrangement configured for needleless jet delivery. In this way, the ingestible capsule device does not include sharp needle points and a mechanism which actuates and retracts the needle is also not required. By inclusion of the burst gate, e.g. provided as a rupturable membrane, it is ensured that drug expelling will only commence once sufficient gas pressure acting on the movable separator is present for carrying out a suitable jet injection.

Existing jet injector systems for jet delivery are known in the art. A skilled person would understand how to select an appropriate jet injector that provides the correct jetting power to deliver the therapeutic substance into the lumen wall, for example from WO 2020/106,750 (PROGENITY INC). Further details and examples are provided further on in the application.

For needle-less jet injection embodiments, the capsule device may be configured to expel drug substance through the nozzle arrangement with a penetration velocity allowing the drug substance to penetrate tissue of the lumen wall.

In other forms of the capsule device, the drug outlet comprises an injection needle wherein the drug substance is expellable through the injection needle.

In further variants the burst gate is operable from a first closed state wherein pressurized gas in the first actuation compartment is not transferred to the second actuation compartment and a second open state wherein pressurized gas flows to the second actuation compartment.

In some forms the movable separator defines a piston arranged for axial slidable movement within a cavity of the capsule housing. The piston may comprise at least one seal for sealing axially between proximal and distal ends of the piston between the actuation chamber and the drug chamber.

In other forms the movable separator comprises a flexible membrane which is separating high-pressure gas in the actuation chamber and the drug substance accommodated in the drug chamber. In some forms the flexible membrane may be provided as a bag or similar enclosure having a single opening at the drug outlet for fluid communication through the drug outlet.

In exemplary embodiments, the capsule device is configured for swallowing by a patient and travelling into a lumen of a gastrointestinal tract of a patient, such as the stomach, the small intestine or the large intestine, respectively. The capsule device may be shaped and sized to allow it to be swallowed by a subject, such as a human.

By the above arrangements an orally administered drug substance can be delivered safely and reliably into the stomach wall or intestinal wall of a living mammal subject.

As used herein, the terms “drug”, “drug substance”, “drug product” or “payload” is meant to encompass any drug formulation capable of being delivered into or onto the specified target site. The drug may be a single drug compound, a premixed or co-formulated multiple drug compound, or even a drug product being mixed by two or more separate drug constituents wherein the mixing is performed either before or during expelling. Representative drugs include pharmaceuticals such as peptides (e.g. insulins, insulin containing drugs, GLP-1 containing drugs as well as derivatives thereof), proteins, and hormones, biologically derived or active agents, hormonal and gene-based agents, nutritional formulas and other substances in both solid, powder or liquid form. Specifically, the drug may be an insulin or a GLP-1 containing drug, this including analogues thereof as well as combinations with one or more other drugs.

### BRIEF DESCRIPTION OF THE DRAWINGS

In the following embodiments of the invention will be described with reference to the drawings, wherein

FIG. 1 is an external perspective view of an ingestible capsule device 100 according to a first embodiment of the invention,

FIG. 2 is a cross-sectional side view of the ingestible capsule device 100 according to the first embodiment of the invention,

FIG. 3 is a cross-sectional side view an ingestible capsule device 200 according to a second embodiment of the invention, and

FIG. 4 provides a top view and a cross sectional side view of a second example burst gate 70 provided as a rupturable disc suitable for use in a gas generator and release arrangement according to the present invention,

FIGS. 5a-5c illustrate schematically a third example burst gate 70′ suitable for use in a gas generator and release arrangement according to the present invention, and

FIGS. 6a-6c illustrate schematically a fourth example burst gate 70″ suitable for use in a gas generator and release arrangement according to the present invention.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The terms “assembly” and “subassembly” do not imply that the described components necessarily can be assembled to provide a unitary or functional assembly or subassembly during a given assembly procedure but is merely used to describe components grouped together as being functionally more closely related.

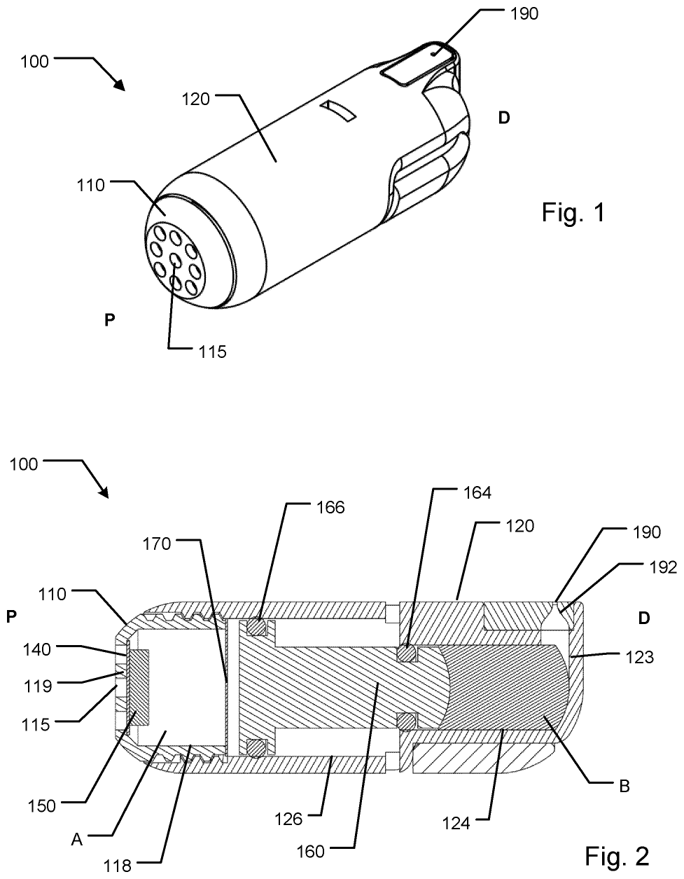

With reference to FIG. 1 a first embodiment of a drug delivery device in accordance with the invention will be described, the embodiment being designed to provide an ingestible capsule device 100 sized and shaped to be ingested by a patient and configured for subsequently being deployed when in a target lumen of the patient so as to cause a dose of a liquid drug to be expelled through a drug outlet provided at an external portion of the capsule device 100. It is to be noted that the disclosed ingestible capsule device 100, in the following referred to simply as “capsule”, is only exemplary and, in accordance with the invention, may be provided in other forms having different capsule outer shapes. Also, although the shown outlet provides an outlet nozzle opening for expelling a substance directly through the outlet, the outlet may be provided in alternative forms, such as having an outlet opening associated with an injection needle. The disclosed embodiment relates to a capsule 100 suitable for being ingested by a patient to allow the capsule to enter a lumen of the Gastro-Intestinal tract, more specifically the small intestine, and subsequently to eject a liquid dose of a payload, such as a drug substance at a target location either inside the lumen, or into tissue of the lumen wall surrounding the lumen. In other embodiments, the capsule may be configured for expelling a substance in other locations of the Gastro-Intestinal system, such as the stomach, the large intestine or even in other lumen parts of a subject.

In the shown embodiment capsule 100, the drug substance is intended to be prepared from or provided as a single drug product. Alternatively, the substance may be prepared from at least two drug products. When the substance is prepared by two drug products, a first product may be stored within a first reservoir whereas a second product may be stored in a second reservoir and mixed prior to expelling or even mixed during expelling through the outlet. In some embodiments, the first drug component is provided initially as a lyophilized drug substance, such as a powder, whereas the second drug component is a reconstitution liquid, such as a diluent. In other embodiments, the two or more drug products are each initially provided as a liquid which are mixed with each other prior to or during drug expelling.

Referring to FIGS. 1 and 2, the capsule 100 includes a multi-part housing having an elongated shape extending along an axis, which is also referred to in the following as “the longitudinal axis”. The elongated housing includes a cylindrical section and further include rounded end portions, i.e. a proximal end portion and a distal end portion. In the shown embodiment an outlet 190 is arranged at a sidewall portion of the cylindrical section, at the distal end of the capsule 100. The outlet thus points radially outwards from a surface arranged to be in close proximity with the tissue of the lumen wall. In the shown embodiment, the capsule is shaped in shape and size to roughly correspond to a 00 elongated capsule.

In the shown embodiment, the capsule 100 includes a drug outlet 190 that is positioned laterally to the longitudinal axis. The outlet 190 may be an aperture to permit jet injection to occur.

Existing jet injector systems for jet drug delivery are known in the art. A skilled person would understand how to select an appropriate jet injector that provides the correct jetting power to deliver the therapeutic substance into the lumen wall 24, for example from WO 2020/106,750 A1 (PROGENITY INC).

In particular, the skilled person would understand that during drug delivery into a GI tract of a patient using jet injection, the jet stream created by the jet injector interfaces the lumen of the GI tract and the surface of the GI tract facing the lumen. Ultimately, the drug substance is deposited into the submucosal and/or the mucosal tissue by the substance impacting the mucosal layer of the GI tract (e.g. the epithelial layer and any mucus that may be present on the epithelial layer) as a stable jet stream of fluid with minimal breakup into a spray.

The volume of fluid of the drug substance experiences a peak fluid pressure that generates the jet stream that exits the jet injector with a peak jet velocity. The jet stream impacts the interface of the lumen of the GI tract and the surface of the GI tract facing the lumen with a peak jet power, peak jet pressure and peak jet force. The skilled person would recognise that these three parameters are interconnected.

The skilled person would understand how to assess and measure the various jet injector characteristics for suitability of use in the described type of jet injection. For example, one way to assess the jet power is to release the jets onto force sensors which measure the force the jet. Based on the force reading, and knowing the area of the nozzle and density of the jetted liquid, the jet velocity can be determined using equation 1. Based on the calculated velocity, the power (in Watts) can be calculated using equation 2. To evaluate the jet pressure (i.e. the pressure at which the jet stream is expelled), equation 3 can be used.

$$F = ρ {AV}^{2} \qquad ( equation 1 )$$

$$P = \frac{1}{2} ρ {AV}^{3} \qquad ( equation 2 )$$

$$V = \sqrt{\frac{2 * {P}_{b a r} * 100000}{ρ * C}} \qquad ( equation 3 )$$

F=Force (N)
ρ=Density (kg/m3)
A=Area of nozzle (m2)
V=Velocity (m/s)
P=power (W)
P_{bar}=Pressure (bar)
C=Nozzle Loss Coefficient (Typically 0.95)

Referring to FIG. 2, the shown multi-part housing includes a first housing portion, i.e. a proximal housing portion 110, arranged at the proximal end, a generally cylindrical sleeve shaped distal housing portion 120 ending at the distal end with a generally rounded end surface. In the shown embodiment the proximal and distal housing portions are fixedly mounted relative to each other by means of a threaded engagement. Other attachment or joining means may be used in other embodiments. A proximal end wall 119 of the proximal housing portion 110 includes a multitude of openings or channels 115 that in combination serve as a fluid inlet which allows ingress of gastrointestinal fluid present in the GI tract towards the interior of the capsule 100.

FIG. 2 shows a cross sectional view of the capsule 100 in an initial state wherein the capsule is ready to be ingested by a patient. Inside capsule 100, at the distal end thereof, a hollow first cylindrical section 124 is arranged extending along the longitudinal axis, this section having a radially inwards facing surface having a first diameter. The first cylindrical section 124 is terminated at the distal end by a distally arranged end face 123. The first cylindrical section 124 extends proximally towards a hollow second cylindrical section 126, coaxially arranged with the first cylindrical section 124 and having a radially inwards facing surface with a larger diameter than the diameter of the first cylindrical section 124. A hollow third cylindrical section 118 extends coaxially with the first and second cylindrical sections 124 and 126 from the second cylindrical section to the most proximal end of the capsule 100 wherein the third cylindrical section 118 is terminated by proximal end wall 119. In the shown embodiment, proximal end wall 119 has a central planar portion.

A piston 160 is arranged for axial slidable movement inside the hollow space provided by the first cylindrical section 124 and the second cylindrical section 126. The piston 160 includes a small diameter section having a circumferential seal 164 that seals against the radially inwards surface of the first cylindrical section 124. The piston 160 further includes a large diameter section having a circumferential seal 166 that seals against the radially inwards surface of the second cylindrical section 124. The piston includes a distal facing circular end surface having a diameter which is made slightly smaller than the diameter of the first cylindrical section 124. At the proximal end of piston 160, the piston includes a proximal facing circular end surface having a diameter slightly smaller than the diameter of the second cylindrical section 126.

When the capsule 100 assumes the initial state, i.e. prior to administration, the piston 160 is disposed in a start position remote from distally arranged end face 123. In this initial state, the circular distal end face of the piston 160, the radially inwards surface of the first cylindrical section 124 and the distally arranged end face 123 in combination defines a reservoir or drug chamber B. A liquid drug substance is accommodated in the drug chamber B. The outlet 190 arranged at the distal end of drug chamber B defines a fluid outlet passage from the reservoir to the exterior of the capsule 100. In the shown embodiment, the outlet 190 includes a jet nozzle 192 dimensioned and shaped to create a liquid jet stream of drug when the drug is forced through the outlet. The reservoir is sealed at the outlet with a seal (not shown) designed to break at an elevated pressure of the liquid drug.

Inside capsule 100, at the proximal end thereof, a drive system is arranged configured for driving the piston 160 towards the outlet 190 upon triggering of the drive system, i.e. upon triggering by a predefined condition. The drive system comprises a gas generator capable of producing a gas for driving forward the piston 160 when elevated gas pressure from the gas generator exceeds a predefined threshold. In the shown embodiment, the gas generator is arranged inside hollow third cylindrical section 118 which forms part of an actuation chamber A.

Gas may be generated by chemical reaction so that, once the gas generator is actuated, gas is produced to form pressurized gas in the actuation chamber A of capsule 100. Different principles may be used for providing gas generation inside the actuation chamber A, for example by using a gas producing cell, such as a hydrogen cell, an airbag inflator, a gas generator utilizing phase change, or a generator which incorporates mixing of reactants to chemically react to form a gas, such as by mixing sodium bicarbonate and acid. For gas generation using mixing of reactants, either all reactants may be stored on board the capsule prior to actuation, or at least one reactant may be introduced into the capsule for mixing with a reactant stored on board the capsule.

The following are examples of chemical reactions which produce carbon dioxide CO2 and which may be used as the components for generating pressurized gas in the actuation chamber A:
Example 1 (calcium carbonate with hydrochloric acid): CaCo3+2HCl→CaCl₂)+H₂O+CO2
Example 2 (citric acid with sodium bicarbonate): C6H8O7+3NaHCO3→3H2O+CO2+Na3C6H5O7
Example 3 (tartaric acid with sodium bicarbonate): H2C4H4O6+2NaHCO3→Na2C4H4O6+2H2O+2CO2

Examples of acids for effervescent reaction:
Citric acid
Acetic acid
Hydrochloric acid
Tartaric acid
Malic acid
Adipic acid
Ascorbic acid
Fumaric acid

Examples of carbonate salts for effervescent reaction:
Sodium bicarbonate
Sodium carbonate
Calcium carbonate
Potassium bicarbonate

In other embodiments, the effervescent reaction may occur by one or more solid state components being wetted (e.g. exposed to intestinal fluid or other fluid stored in capsule 100) which causes the effervescent reaction.

In capsule 100 shown embodiment in FIG. 2, gas is generated in the actuation chamber A by means of an internally arranged effervescent material 150 arranged in the actuation chamber, and by means of a semipermeable membrane 140 which serves to introduce gastrointestinal fluid into the actuation chamber A to react with the effervescent material portion 150.

Effervescent material portion 150 formed from powder components that are subsequently compressed into block-shape includes an effervescent couple comprised of at least one acidic material and one basic material, such as sodium bicarbonate and citric acid. The block of effervescent material 150 is adhered to semipermeable membrane 140 to ensure close proximity with the membrane while leaving a volume of actuation chamber A available for gas generation.

As noted above the proximal housing portion 110, and more specifically the central planar portion of proximal end wall 119 includes a multitude of openings or channels 115 arranged at the proximal end face which allows ingress of gastrointestinal fluid into the actuation chamber A. The semi-permeable membrane 145 is arranged with its proximally facing surface in intimate contact with the distal facing surface of the central planar portion of proximal end wall 119. Hence, gastrointestinal fluid that enters the capsule 100 needs to pass through the openings 115 and the semi-permeable membrane 145. The central planar portion of proximal end wall 119 provides sufficient rigidity to serve as a backing or support for the semi-permeable membrane 145 when pressure builds up in the actuation chamber A.

For the shown embodiment capsule 100 example materials for the semipermeable membrane 140 may be made from Standard Grade Regenerated Cellulose (RC). The material for the semipermeable membrane 140 may be selected so that it is biodegradable when subjected to biological fluid.

A burst member serving as a burst gate is arranged axially between the actuation chamber A and the piston 160. The burst member functions as a gate to release mechanical load provided by the pressurized gas onto the piston 160 but only upon increase in gas pressure in the actuation chamber A above a predefined threshold pressure level. For gas pressures below the predefined threshold pressure level, the burst member forms a substantially gas tight seal preventing the piston from receiving mechanical load from the gas which would otherwise cause the piston 160 to move towards the outlet.

In the shown embodiment capsule 100 includes a burst gate in the form of a rupturable membrane 170 which is mounted axially fixed at an axial location adjacent to the piston 160 in its initial position, i.e. its start position. Different attachment methods may be used for mounting the rupturable membrane 170 in capsule 100, such as by being adhered relative to a housing portion, or by clamping of the burst membrane between rigid structures mounted fixedly relative to one or more housing portions.

In the FIG. 2 embodiment, the rupturable membrane 170 is formed as a thin planar disc-shaped membrane. Example materials for the rupturable membrane may be selected from a metallic material, such as aluminium, a polymer material or other suitable material that will exhibit a well-defined ability to burst at the predetermined threshold pressure level. Instead of forming the rupturable membrane as a planar disc, the burst gate may include forms of thin-layered material which in the initial state may exhibit or comprise one or more convex and/or concave portions.

In the example capsule 100 shown in FIGS. 1 and 2, the jet delivery may be dimensioned to operate at a liquid pressure in the order of 18 bar in the drug chamber B. In the example shown the semi-permeable membrane 140 will be able to withstand a maximum gas pressure slightly above 8 bar before leaking. In accordance herewith, the burst disc 170 may be designed to provide a release of gas towards the piston when the gas pressure level exceeds 8 bar. However, for the piston 160 used in this embodiment, due to the difference in cross-sectional area of the proximal facing circular end surface relative to the cross-sectional area of the distal facing circular end surface of the piston 160 the liquid pressure in the actuation chamber is magnified to around 18 bars in the drug chamber B, i.e. meeting the targeted fluid pressure in drug chamber B.

The rupturable membrane 170 may in different embodiments include scoring lines or other weakened portions which define the location or locations wherein the rupturable membrane will initiate breaking when gas pressure exceeds the predetermined threshold pressure level.

For capsule device 100, although not visible in FIGS. 1 and 2, the openings 115 are covered by a pH-sensitive enteric coating which initially blocks fluid ingress through the openings 115. As known in the art, the enteric coating may be configured to utilize the marked shift in pH-level that the capsule 100 experiences when travelling from the stomach to the small intestine. After being exposed to gastrointestinal fluid for a specified duration the enteric coating will be degraded to a degree which allows the gastrointestinal fluid to contact the semipermeable membrane 140 and start migration of fluid through the membrane towards the effervescent material portion 150.

For the shown embodiment in FIG. 2, the enteric coating forms a trigger arrangement for actuating the gas generator formed by the semi-permeable membrane 140 and the effervescent material portion 150.

Next the operation of capsule 100 will be described. Subsequent to a patient or user swallows capsule 100, upon entering the small intestine, the enteric coating of the capsule 100 will begin dissolving and gastric fluid will soon after be available through openings 115 enabling fluid transport across the semi-permeable membrane 145.

As fluid gets into contact with the effervescent material portion 150 pressurized gas will start to form in the actuation chamber A whereby gas pressure will gradually increase and provide an increasing mechanical load on the rupturable membrane 170. After lapse of a certain time period the gas pressure level in actuation chamber A exceeds the predetermined threshold pressure level which will cause the rupturable membrane 170 to burst. Hence pressurized gas will flow towards the proximal facing end surface of piston 160 whereby mechanical load will be exerted for moving the piston distally towards the outlet. Due to the difference in cross-sectional area of the proximal facing circular end surface relative to the cross-sectional area of the distal facing circular end surface of the piston 160 the pressure in the actuation chamber is magnified to the hydraulic pressure in the drug chamber B and the drug substance is thrust out through the jet nozzle 192.

Eventually, the piston 160 will bottom out relative to distally arranged end face 123 and the jet stream of drug through the jet nozzle 192 will end. After delivery of the drug substance, the capsule 100 is allowed to pass the alimentary canal and be subsequently excreted.

As an alternative to the shown rupturable membrane 170, a second example burst gate 70 not being planar is depicted in FIG. 4. In this embodiment, a generally disc-shaped valve member made from a thin aluminium sheet is formed with a centrally located concave portion 71a that faces the actuation chamber. The concave central portion 71a connects to a circular connection portion 71b by way of a sharply bent region. A peripheral portion 71c of the valve member connects to the radially outwards portion of connection portion 71b and forms an annular clamping region that is clamped in between two ring shaped mounting structures 72a/72b. Mounting structures 72a/72b are intended for mounting the burst gate 70 relative to the housing portions of the capsule. The shown burst gate 70 is configured for becoming teared along the circular interface between annular band 71c and annular connection portion 71b, i.e. radially inwards to the clamping region, when exerted to excess differential pressure above a predefined burst pressure level.

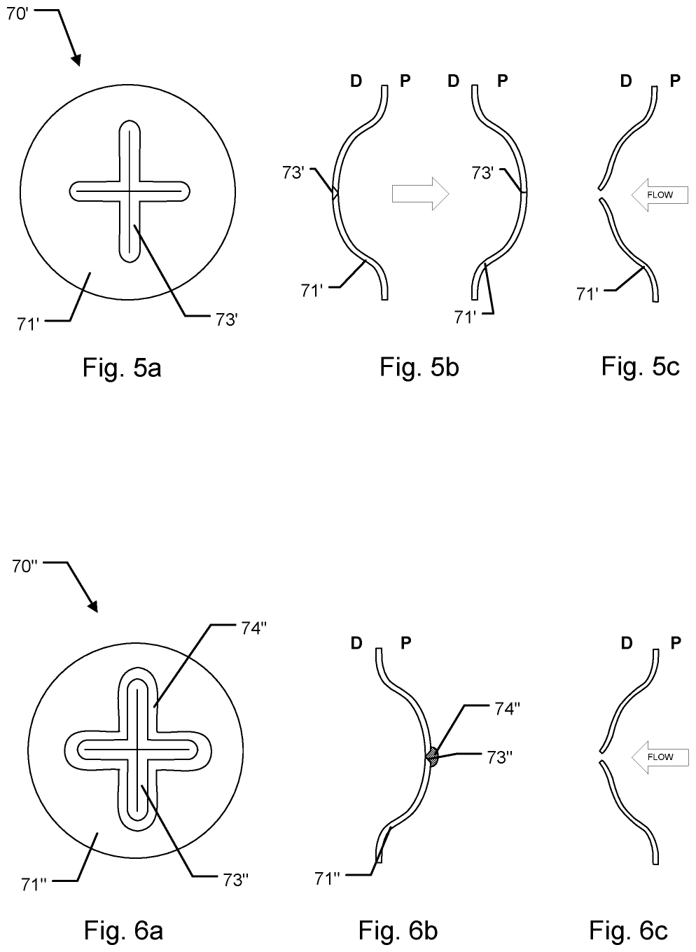

Also, in further embodiments, the burst gate of the capsule device may be provided as a burst valve configured for substantially preventing gas transport across the burst gate until a predefined threshold pressure difference is reached across the burst gate. Referring to FIGS. 5a-5c, a third example burst gate 70′, is formed as a valve member which includes a moulded bistable dome 71′, e.g. a bi-stable spring element, that is moulded on its distal side with a centrally located notched region 73′, the notched region forming a cross when viewed from the downstream side, i.e. on the distal facing surface of burst gate 70′ when mounted in capsule device 100. When the bistable dome 71′ assumes a first stable state (shown in the left-hand side of FIG. 5b) the notched region may either define a sealed opening or a non-sealing opening. When moved to the second stable state shown in the right-hand side of FIG. 5b, the notched region 73′ will either remain sealed or become sealed. Referring to FIG. 5c, upon excessive pressure differential across the burst gate 70′, the bistable dome 71′ will return to the first stable state thereby creating a flow opening at the notched region 73, i.e. a gas port, causing gas transport across the burst valve to be established.

Referring to FIGS. 6a-6b, a fourth example burst gate 70″, is formed as a valve member which again includes a moulded bi-stable dome 71″. Bi-stable dome 71″ is moulded on its proximal side with a centrally located notched region 73″, the notched region forming a cross when viewed from the upstream side, i.e. on the proximal facing surface of burst gate 70″ when mounted in capsule device 100. When the bistable dome 71″ assumes a first stable state (shown in FIG. 6b) the notched region 73″ define an opening along the cross. However, a sealing compound 74″ is arranged in the notched region 73″. and in overlapping relationship with areas adjacent the notched region 73″ so that the sealing compound effectively provides a seal. In different embodiments, the sealing compound may either be provided as a brittle material or a material that is elastically deformable. Referring to FIG. 6c, upon excessive pressure differential across the burst gate 70′, the bistable dome 71″ will move into a second stable state thereby abruptly destroying the seal provided by sealing compound 74″ at the notched region 73″, hence causing gas transport through the gas port to be to be established.

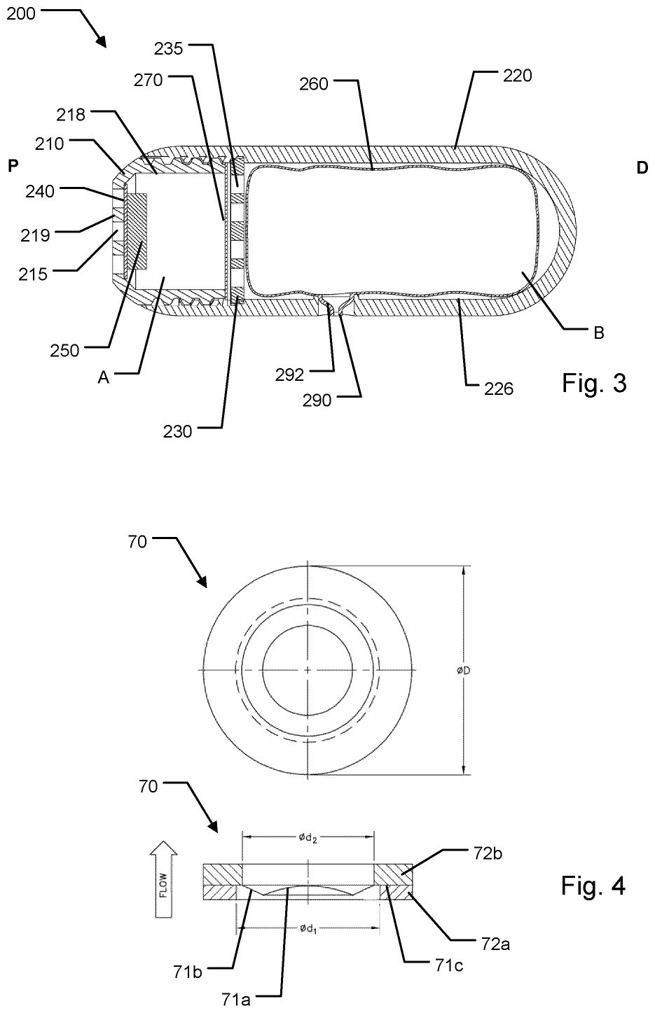

Referring now to FIG. 3, a second embodiment of a capsule 200 will now be described. The capsule 200 corresponds in many aspects to the capsule 100 but the drug chamber B and the expelling mechanism is different. Whereas the capsule 100 relies on a movable separator between the actuation chamber and the drug chamber being provided as a slidable piston 160, the capsule 200 utilizes a flexible membrane 260 separating the actuation chamber A and the drug chamber or reservoir B, whereby the flexible membrane 260 serves as a movable separator.

Capsule 200 again includes a proximal housing portion 210 and a distal housing portion 220. In capsule 200, the trigger arrangement formed by an enteric coating is again triggerable for actuating the gas generator formed by the semi-permeable membrane 240 and the effervescent material portion 250.

The outlet 290 including jet nozzle 292 is located at a side portion of the cylindrical shaped sleeve of capsule 200, arranged approximately midways between the distal end and the proximal end of the capsule 200.

A major portion of the distal housing portion 220 includes a hollow cylindrical section 226, which may be referred to “output cylindrical section” which serves as a space for accommodating the drug reservoir/chamber B. A flexible gas-tight and fluid-tight membrane 260 is arranged within cylindrical section 226. The membrane forms a drug reservoir/chamber B, i.e. configured as a bag and forming an enclosure for the drug substance with a single opening arranged at the outlet 290.

A partitioning wall 230 separates the third cylindrical section 218 and the second cylindrical section 226, the partitioning wall including a plurality of through-going apertures 235 which allow pressurized gas to flow from the third cylindrical section 218 to the second cylindrical section 226.

In FIG. 3, which shows the capsule 200 in the initial state wherein the capsule is ready to be ingested by a patient, the membrane 260 assumes an expanded configuration wherein the bag defined by the membrane takes up a major portion of the cylindrical section 226.

Subsequent to a patient or user swallows capsule 200, upon entering the small intestine, the enteric coating of the capsule 200 will begin dissolving and gastrointestinal fluid will soon after be available through openings 215 to enable fluid transport across the semi-permeable membrane 240.

As fluid gets into contact with the effervescent material portion 250 pressurized gas will start to form in the actuation chamber A whereby gas pressure will gradually increase and provide an increasing mechanical load on the rupturable membrane 270. Upon lapse of a certain time period the gas pressure level in actuation chamber A exceeds the predetermined threshold pressure level which will cause the rupturable membrane 270 to burst.

Hereafter the pressurized gas within actuation chamber A will escape through apertures 235 of partitioning wall 230 whereby pressurized gas will flow from the input cylindrical section 218 to the output cylindrical section 226. Puncture of rupturable membrane 270 will rapidly increase the gas pressure of the output cylindrical section 226 which exerts a mechanical load onto membrane 260 causing the volume of membrane 260, i.e. the drug chamber B, to become smaller. Due to the reduction in volume within the membrane bag 260 the drug substance accommodated in drug reservoir/chamber B is thrust out through the jet nozzle 292.

Eventually, the membrane 260 will assume a collapsed configuration when the pressurized gas has evacuated substantially all of the drug substance accommodated in drug chamber B and the jet stream of drug through the jet nozzle 292 will end. After delivery of the drug substance, the capsule 200 is allowed to pass the alimentary canal and be subsequently excreted.

As described in the above embodiments, subsequent to swallowing, the capsule device first moves through the stomach and subsequently enters the small intestine. Due to the enteric coating becomes dissolved when entering the small intestine the fluid ingress into capsules 100 and 200 will only be initiated upon the enteric coating becoming sufficiently dissolved for fluid ingress through the fluid inlet/semi-permeable membrane is enabled.

An enteric coating may be any suitable coating that allows the coated object to be activated for release in the intestine. In some cases, an enteric coating may dissolve preferentially in the small intestine as compared to the stomach. In other embodiments, the enteric coating may hydrolyse preferentially in the small intestine as compared to the stomach. Non-limiting examples of materials used as enteric coatings include methyl acrylate-methacrylic acid copolymers, cellulose acetate succinate, hydroxy propyl methyl cellulose phthalate, hydroxy propyl methyl cellulose acetate succinate (i.e., hypromellose acetate succinate), polyvinyl acetate phthalate (PVAP), methyl methacrylate-methacrylic acid copolymers, and sodium alginate, and stearic acid. Additional examples are disclosed in e.g. US 2018/0193621 hereby incorporated by reference. A given object (here: capsule), or a fluid inlet only, may be coated with an enteric coating. The enteric coating may be composed to be soluble at a given pH or within a given pH range, e.g. at a pH greater than 5.5, at a pH greater than 6.5, within a range of about 5.6 to 6 or within a range of about 5.6 to 6.5 or 7. The dissolution time at an intestinal pH may be controlled or adjusted by the composition of the enteric coating. For example, the dissolution time at an intestinal pH may be controlled or adjusted by the thickness of the enteric coating.

In other embodiments, the condition for controlling when triggering is to occur may be provided by means of other principles. For example, a dissolvable layer may be disposed initially blocking the fluid inlet of the capsule, with dissolution of the dissolvable layer being initiated at first exposure to gastric fluid, and with the timing of the dissolvable layer being decisive for the location at which the capsule deploys. Also, such as for a stomach deployable capsule, no coating may be present, so that the triggering of the gas generator occurs as soon as sufficient liquid has been transferred through the semi-permeable membrane. Still other triggering principles may rely on temperature change induced passage of gastric fluid though the fluid inlet and into the capsule gas generator.

Although the above description of exemplary embodiments mainly concern ingestible capsules for delivery in the small intestine, the present invention generally finds utility in capsule devices for lumen insertion in general, wherein a capsule device is positionable into a body lumen for delivery of a drug product. Non-limiting examples of capsule devices include capsule devices for delivery in the stomach or delivery into the tissue of the stomach wall. For example, various self-righting or self-orienting structures and/or methods described in WO 2018/213,600 A1 can be employed by the capsule device in accordance with the present disclosure. WO 2018/213,600 A1 is incorporated herein by reference in its entirety.

In various embodiments of capsules utilizing the gas generation and release arrangement described herein, drug delivery may be performed using a delivery member, such as a needle, via a jet stream of liquid to provide needle-free liquid jet penetration into the mucosal lining or via spraying inside the lumen. In still other embodiments, the inventive gas generation release arrangement set forth in this disclosure may be used to trigger delivery of a solid drug pellet which is to be inserted into a lumen wall.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A capsule device suitable for insertion into a lumen, such as a gastrointestinal lumen, of a human or animal subject, wherein the capsule device comprises:
a capsule housing,
a drug chamber configured to accommodate a drug substance, the drug chamber leading to a drug outlet,
an actuation chamber,
a movable separator arranged between the actuation chamber and the drug chamber, wherein movement of the movable separator expels drug substance from the drug chamber through the drug outlet, and
a gas generator configured actuatable to generate pressurized gas in the actuation chamber for exerting load on the movable separator to expel the drug substance,
wherein a burst gate is arranged between the gas generator and the movable separator, the burst gate being configured to release load onto the movable separator upon increase in gas pressure in the actuation chamber above a threshold pressure level to thereby initiate expelling of the drug substance.

2. The capsule device as in claim 1, wherein the actuation chamber comprises first and second actuation compartments being separated by the burst gate, wherein the gas generator supplies gas to the first actuation compartment, and wherein the second actuation compartment is in gas fluid communication with the movable separator.

3. The capsule device as in claim 1, wherein the burst gate comprises a rupturable membrane, such as a burst disc.

4. The capsule device as in claim 1, wherein the burst gate comprises a burst valve arrangement.

5. The capsule device as in claim 4, wherein the burst valve arrangement comprises a bi-stable spring element being movable from a first stable position to a second stable position upon an increase in gas pressure in the first actuation compartment, wherein the bi-stable spring element releases mechanical load onto the movable separator when moving from the first stable position towards the second stable position.

6. The capsule device as in claim 5, wherein the bi-stable spring element comprises a gas port which, when the bi-stable element assumes the first stable position, is maintained closed by a brittle and/or breakable material portion, and wherein the brittle and/or breakable material portion breaks when the bi-stable element is moved towards the second stable position to enable pressurized gas to flow through the gas port.

7. The capsule device as in claim 1, wherein the gas generator comprises a trigger arrangement configured to actuate the gas generator.

8. The capsule device as in claim 7, wherein the trigger arrangement comprises a GI tract environmentally-sensitive mechanism comprising a trigger member, wherein the trigger member is characterised by at least one of the group comprising:
a) the trigger member comprises a material that degrades, erodes and/or dissolves due to a change in pH in the GI tract;
b) the trigger member comprises a material that degrades, erodes and/or dissolves due to a pH in the GI tract;
c) the trigger member comprises a material that degrades, erodes and/or dissolves due to a presence of an enzyme in the GI tract; and
d) the trigger member comprises a material that degrades, erodes and/or dissolves due to a change in concentration of an enzyme in the GI tract.

9. The capsule device as in claim 1, wherein the actuation chamber comprises effervescent material, wherein the capsule housing comprises a fluid inlet portion leading to the effervescent material.

10. The capsule device as in claim 9, wherein the fluid inlet portion initially comprises an enteric coating adapted to dissolve when subjected to a biological fluid within the lumen, wherein biological fluid within the lumen is allowed to flow through the fluid inlet portion upon dissolving of the enteric coating to cause contact between the biological fluid and the effervescent material.

11. The capsule device as in claim 9, wherein the fluid inlet portion comprises a semi-permeable membrane, allowing biological fluid within the lumen to migrate through the semi-permeable membrane and enter into contact with the effervescent material.

12. The capsule device as in claim 1, wherein the capsule device comprises a liquid compartment filled with a liquid, wherein the gas generator comprises an effervescent material configured to generate gas when subjected to contact with liquid from the liquid compartment, and wherein operation of the trigger arrangement enables contact between the effervescent material and the liquid.

13. The capsule device as in claim 1, wherein the gas generator comprises a first reactant and a second reactant configured to generate gas upon contact between the first reactant and the second reactant.

14. The capsule device as in claim 1, wherein the movable separator comprises a piston arranged for slidable movement in the drug chamber.

15. The capsule device as in claim 1, wherein the capsule device is configured for insertion into a lumen having a lumen wall, such as a gastrointestinal lumen wall, of a human or animal subject, wherein the drug outlet comprises a jet nozzle arrangement configured for needleless jet delivery, and wherein the capsule device is configured to expel the drug substance through the jet nozzle arrangement with a penetration velocity allowing the drug substance to penetrate tissue of the lumen wall.
